(19) United States
(12) Patent Application Publication
(10) Pub. No.: US 2003/0186338 A1
(43) Pub. Date: Oct. 2, 2003

(54) Marker for diagnosing breast cancers and ovarian cancers

(76) Inventor: Walter A. Deutsch, Baton Rouge, LA (US)

Correspondence Address: PATENT DEPARTMENT, TAYLOR, PORTER, BROOKS & PHILLIPS, L.L.P, P.O. BOX 2471, BATON ROUGE, LA, 70821-2471, US

(21) Appl. No.: 10/375,611

(22) Filed: Feb. 27, 2003

Related U.S. Application Data
(60) Provisional application No. 60/360,488, filed on Feb. 28, 2002.

(51) Int. Cl. G01N 33/574; C07K 16/30

(52) U.S. Cl. 435/7.23; 530/388.8

(57) ABSTRACT

The protein APE/ref-1 is not phosphorylated in breast cancer cells. By contrast, phosphorylated APE/ref-1 is found in normal breast cells. Although it is not known why APE/ref-1 is not phosphorylated in breast cancer cells, the absence of phosphorylated APE/ref-1 in breast cancer cells allows it to be used as a marker to diagnose breast cancers, and to monitor their progress. Monitoring the phosphorylation status of APE/ref-1 provides a simple, straightforward means to determine the onset and progression of breast cancer. It is expected that the same marker will also be useful in determining the onset and progression of ovarian cancer.

The benefit of the Feb. 28, 2002 filing date of provisional application serial No. 60/360,488 is claimed under 35 U.S.C. § 119(e).

The development of this invention was partially funded by the Government under grant number R01 ES07815 awarded by the National Institutes of Health, and under a subcontract under grant number P01 CA 75426-03 awarded by the National Institutes of Health. The Government has certain rights in this invention.

This invention pertains to a marker for diagnosing breast cancers and ovarian cancers, as well as to methods and compositions useful in such diagnoses.

An important enzyme in the repair of DNA is that known as APE/ref-1. (“APE” stands for apurinic/apyrimidinic (AP, or baseless) endonuclease; and “ref” denotes “redox effector.”) APE/ref-1 plays a critical role in the repair of potentially mutagenic DNA sites in which a base has been deleted (for example, by a mutagen or by a DNA copying error. APE/ref-1 also has another, very different function: namely, it controls the oxidation-reduction (or redox) state of several important DNA transcription factors, including those known as FOS, JUN, HIF-1α, PAX-8, and NF-κB. Surprisingly, APE/ref-1 also plays a role in converting the protein p53 from an inert form to an active form in the control of apoptosis and cell-cycle control. Mis-regulation of p53 has been implicated in several types of cancer.

The different activities of APE/ref-1 appear to be controlled by factors that direct the protein either to DNA repair, or to the regulation of DNA transcription. Our research group has previously reported that APE/ref-1 is phosphorylated in vivo by PKC (protein kinase C). The phosphorylated form of APE/ref-1 then has substantially increased redox activity. Put differently, the phosphorylated form of APE/ref-1 controls transcription, while the un-phosphorylated form is involved in DNA repair. See A. Yacoub et al., “The DNA repair activity of human redox/repair protein APE/Ref-1 is inactivated by phosphorylation,” Cancer Res., vol. 57, pp. 5457-5459 (1997); and M. Hsieh et al., “Activation of APE/ref-1 redox activity is mediated by reactive oxygen species and PKC phosphorylation,” Nucleic Acids Res., vol.29, pp. 3116-3122 (2001).

There have been several prior studies reporting altered expression of APE/ref-1 in various cancers. For example, S. Kakolyris et al., “Human AP endonuclease 1 (HAPI) protein expression in breast cancer correlates with lymph node status and angiogenesis,” Br. J. Cancer, vol. 77, pp. 1169-1173 (1998) reported that, in regions of invasive tumor necrosis as well as in lactating mammary glands, APE/ref-1 was found predominantly in the cytoplasm, instead of its usual predominant location in the cell nucleus. A similar mis-localization has also been reported in epithelial ovarian cancers. See D. Moore et al., “Alterations in the expression of the DNA repair/redox enzyme APE/ref-1 in epithelial ovarian cancers,” Clin. Cancer Res., vol. 6, pp. 602-609 (2000). Each of these two papers also described earlier work that had reported similar patterns in adenomas and carcinomas of the colon.

K. Robertson et al., “Downregulation of apurinic/apyrimidinic endonuclease expression is associated with the induction of apoptosis in differentiating myeloid leukemia cells,” Cell Growth & Different., vol. 8, pp. 443-449 (1997) reported that APE/ref-1 expression was down-regulated in a myeloid leukemia cell line after the cells had been stimulated to differentiate and undergo apoptosis.

M. Kelley et al., “Elevated and altered expression of the multifunctional DNA base excision repair and redox enzyme Ape1/ref-I in prostate cancer,” Clin. Cancer Res., vol. 7, pp. 824-830 (2001) reported that APE/ref-1 is overexpressed in prostate cancer, particularly in the cytoplasm. Kelley et al. stated at page 828 (citations omitted):

“Furthermore, we haven't determined whether the Ape1/ref-1 found in the various prostate samples is phosphorylated or not. Recent data have emerged showing that Ape1/ref-1 is modified posttranslationally by phosphorylation; however, there is a disagreement on whether the phosphorylation of Ape1/ref-1 affects the repair or redox function of the protein. Nevertheless, this will be an important issue to clarify, not only for mechanistic studies but also to develop Ape1/ref-1 antibodies that will be able to determine the phosphorylation status of Ape1/ref-1 in prostate cancer and its impact on Ape1/ref-1 function in these cells.”

U.S. Pat. No. 5,919,643 discloses that premalignant or malignant conditions in humans are correlated with elevated levels of apurinic/apyrimidinic endonucleases. At column 48, lines 9-27 of this patent appears the following paragraph:

“Various cell lines containing wild-type or natural or engineered mutations in APE can be used to study various functional attributes of APE and how a candidate compound affects these attributes. Methods for engineering genetic constructs are described elsewhere in this document. In such assays, the compound would be formulated appropriately, given its biochemical nature, and contacted with a target cell. Depending on the assay, culture may be required. The cell may then be examined by virtue of a number of different physiologic assays. These might include measurement of cell growth, division, contact inhibition, metastasis, soft agar formation or other characteristic. Alternatively, molecular analysis may be performed in which the molecular function or state of APE, or related pathways, may be explored. This may involve assays such as those for protein expression, enzyme function, substrate utilization, phosphorylation states of various molecules including APE, cAMP levels, mRNA expression (including differential display of whole cell or polyA RNA) and others described above.”

U.S. Pat. No. 6,046,036 discloses DNA sequences encoding fusion proteins having two DNA repair proteins fused to one another. It was said that when such fusion proteins were expressed in cells, cell survival should be greater than would result from the expression of either component of the fusion protein alone. Among several possible DNA repair proteins mentioned for possible use in such a fusion protein was APE. FIG. 22 of this patent displays phosphorylation sites for human APE.

I have discovered that APE/ref-1 is not phosphorylated in breast cancer cells. Phosphorylated APE/ref-1 was absent from each of two breast cancer cell lines assayed to date. By contrast, phosphorylated APE/ref-1 was found in usual concentrations in a normal, control breast cell line. Although it is not known why APE/ref-1 is unphosphorylated in breast cancer cells, the absence of phosphorylated APE/ref-1 in breast cancer cells allows it to be used as a marker to diagnose breast cancers, and to monitor the progress of the cancers. Thus monitoring the phosphorylation status of APE/ref-1 provides a simple, straightforward means to determine the onset and progression of breast cancer, similar to the PSA test that is now employed for monitoring prostate cancer in men. It is expected that the same marker will also be useful in determining the onset and progression of ovarian cancer.

### EXAMPLE 1

Absence of Phosphorylated APE/ref-1 from MCF7 Breast Cancer Cells

This finding was most unexpected. In the course of work that was originally directed to investigating the effect of APE/ref-1 phosphorylation on redox activity, I examined APE/ref-1 phosphorylation levels in several different cell types. The purpose of these examinations was to determine whether certain redox observations were cell-specific. One of these tests was conducted on the breast cell cancer line MCF7, which was a kind gift from Dr. Mark Kelley, The Wells Center, Indiana University School of Medicine. Surprisingly, no phosphorylated APE/ref-1 was detected in these cells, even though Western blot analysis verified that the MCF7 cells contained ample levels of the APE/ref-1 protein (data not shown).

### EXAMPLES 2 and 3

Absence of Phosphorylated APE/ref-1 from Mda231 Breast Cancer Cells, and its Presence in Normal Breast Cells

A second breast cancer cell line was then tested with similar results, namely line Mda231, also a kind gift from Dr. Mark Kelley. When a normal breast cell line was tested for comparison (HTB125, obtained from the American Type Culture Collection), phosphorylation levels for APE/ref-1 were found to be normal. These initial observations led to the discovery that the absence of phosphorylated APE/ref-1 is a marker for breast cancer. In view of the importance of APE/ref-1 to the survival of normal cells, the absence of the phosphorylated form of APE/ref-1 in breast cancer cells could have profound negative consequences for a patient.

### EXAMPLE 4

Overexpression and Radiolabelling of APE/ref-1

APE/ref-1 has been prepared by overexpression with an inducible promoter, generating a pure protein containing roughly 20,000 cpm of ³²P-label, following the methodology of M. Hsieh et al., “Activation of APE/Ref-1 redox activity is mediated by reactive oxygen species and PKC phosphorylation,” Nucleic Acids Res, vol. 29, pp. 3116-3122 (2001). Briefly, wild-type 293 human cells (a kind gift from Dr. J. Ye, Pennington Biomedical Research Center, Baton Rouge, La.) were seeded at 2×10⁶ cells/mL. The 293 cells were transformed to transiently express APE/ref-1. The cells were then treated with 130 nM hypochloric acid (HOCl) for 12 hours, followed by 200 μM methyl methanesulfonate for 1 hour. The cells were also labeled with inorganic ³²P. Following treatment, the cells were washed twice with phosphate-buffered saline, and were then harvested by scraping them off the plate. Cells were resuspended in 200 μL of mild lysis buffer (1% NP-40; 150 mM NaCl; 10 mM Tris; pH 7.2; 2 mM EDTA; 50 mM NaF; 0.2 mM sodium vanadate; 100 U/mL Aprotinin; 10 μg/mL leupeptin; and a 100-fold dilution of Phosphatase Inhibitor cocktail, Sigma), and were then incubated on ice for 20 minutes. Particulates were removed by centrifugation at 15,000 rpm for 30 minutes at 4° C. Supernatants were collected, and protein amounts were determined by the Bradford method. The resulting cell lysates (400 μg) were diluted to a final volume of 100 μL with TETN250 buffer (25 mM Tris-HCl, pH 7.5, 5 mM EDTA, 250 mM NaC, and 1 % Triton X-100), and were pre-cleared with Protein A Sepharose (Zymed, San Francisco, Calif.) for 1 hour at 4° C. with agitation. A monoclonal antibody against APE/Ref-1 (Novus Biologicals, Littleton, Colo.; 1:200 in 100 μL TETN250 with 5% w/v BSA (Boehringer Mannheim)) was then added to the pre-cleared lysate, which was then incubated overnight at 4° C. with agitation to form an immunocomplex. Protein A was added to the mixture, which was then incubated an additional 3 hours at 4° C. with agitation to form a Protein A-immunocomplex conjugate. The Protein A-immunocomplex conjugate was then collected by centrifugation at 10,000 g for 20 seconds. The pellet was washed once with TETN250 and once with TE buffer (10 mM Tris-HCl, pH 7.5, and 5 mM EDTA). Sodium dodecylsulfate (SDS) gel-loading buffer (20 μL, 50 mM Tris, pH 6.8, 2% SDS, 0.1% bromophenol blue, and 10% glycerol) was added to the pellet, and the mixture was then boiled for 3 minutes. After centrifugation at 10,000 g, the supernatant was transferred to a fresh tube. The supernatant (20 μL) was electrophoresed on a 12% SDS-polyacrylamide gel. After electrophoresis the immunoprecipitated hAPE was extracted from the gel and the amount of ³²P determined.

### EXAMPLE 5

Determination of APE/ref-1 Phosphorylation Sites

Although it is believed that the phosphorylation of APE/ref-1 is most likely attributable to PKC, it is also possible that another kinase may be involved. The data are suggestive of PKC because experimental conditions were chosen where PKC was up-regulated. However, the phosphorylation status of APE/ref-1 may be used as a cancer marker as described here, regardless of whether PKC or another kinase induces the phosphorylation.

The APE/ref-1 sites subject to phosphorylation by PKC will be determined. Preliminary analysis of protein motifs using GeneRunner software (version 3.02, Hastings Software, Hastings, N.Y.) and the PROSITE database identified 5 probable sites for PKC-induced phosphorylation of APE/ref-1: amino acids 56 and 61 (in the redox domain), and amino acids 123, 201, and 252 (in the DNA repair domain). Each of these sites is a serine, except that site 61 is a threonine.

Using different software, NetPhos (version 2.0, Center for Biological Sequence Analysis, Technical University of Denmark), phosphorylation sites were instead predicted at serine 26 (in the nuclear localization signal sequence); serines 54, and 56 (in the redox domain); and serines 123, 201, and 298 (in the DNA repair domain). Other predicted phosphorylation sites were threonines at 233 and 260, and tyrosines at 45, 128, 257, and 262. The single most probable phosphorylation site identified by this software was serine 26.

Experimental confirmation (or correction) of the phosphorylation sites will be carried out by matrix-assisted laser desorption ionization mass spectrometry (MALDI-MS) of immunoprecipitated, ³²P-labeled, phosphorylated protein. Preliminary MALDI-MS results, which need to be confirmed, suggested that sites 26 and 252 were phosphorylated.

Without wishing to be bound by this theory, it is hypothesized that phosphorylation at site 26 could be an important event in directing APE/ref-1 from the cytoplasm to the nucleus, because that site is within the nuclear localization signal sequence (amino acid residues 1-36). Lack of phosphorylation may confine the protein to the cytoplasm, and thus explain the mis-localization of the protein in breast cancer as previously noted.

As used in the specification and claims, all amino acid position numbers are in accordance with those shown in A. Yacoub et al., “The DNA repair activity of human redox/repair protein APE/Ref-1 is inactivated by phosphorylation,” Cancer Res., vol. 57, pp.5457-5459 (1997); and GenBank accession number M92444, which gives the following inferred amino acid sequence for human APE/Ref-1 (SEQ ID NO: 1):

MPKRGKKGAVAEDGDELRTEPEAKKSKTAAKKNDKEAAGEGPALYEDP
PDQKTSPSGKPATLKICSWNVDGLRAWIKKKGLDWVKEEAPDILCLQET
KCSENKLPAELQELPGLSHQYWSAPSDKEGYSGVGLLSRQCPLKVSYG
IGDEEHDQEGRVIVAEFDSFVLVTAYVPNAGRGLVRLEYRQRWDEAFR
KFLKGLASRKPLVLCGDLNVAHEEIDLRNPKGNKKNAGFTPQERQGFGE
LLQAVPLADSFRHLYPNTPYAYTFWTYMMNARSKNVGWRLDYFLLSHS
LLPALCDSKIRSKALGSDHCPITLYLAL

Once the phosphorylation sites have been identified, site-directed mutagenesis of each such site (using standard techniques known in the art) will be conducted, and the site-mutated protein will then be expressed in human cells in culture, to confirm that the mutated protein is not phosphorylated (or at least is not phosphorylated to the same extent) in vivo, thereby confirming that each identified site in the unmutated protein is a phosphorylation site.

Several site-directed mutants of APE/ref-1 have been prepared to date, with mutations in both domains of the APE/ref-1 molecule. Most of the site-specific mutations to date have been specific changes to cysteine residues (amino acids 65, 296, 99, and 310) to identify locations important to the redox activity of the protein. Also, mutations have been made at probable phosphorylation positions 56 and 61, both lying within the redox portion of APE/ref-1. Mutations at other probable phosphorylation positions in the nuclear localization sequence, repair, and redox domains will likewise be generated.

The site-directed mutations were generated with a Stratagene Quick Change Site-Directed Mutagenesis kit, using the manufacturer's recommended protocols, except that the elongation time was 12 minutes at 68° C., and the reaction was continued for 16 cycles. The PCR primers used encoded alanine at the positions to be altered, because alanine is not susceptible to ordinary phosphorylation. The template for the PCR reaction was pGex-3× APE/ref-1. (The vector pGex-3× is a fusion-protein expression vector available commercially from Pharmacia (Peapeck, N.J.). It combines glutathione S-transferase and the protein of interest, in this case APE/ref-1, via a small spacer region that is susceptible to Factor X cleavage.)

All site-directed mutation constructs will be placed in the fusion protein expression vector pGex4T (Pharmacia; pGex4T is an updated version of the vector pGex-3× previously described), which will be used to overexpress the encoded sequence as a fusion protein with glutathione S-transferase in K562 myeloid leukemia cells (also a kind gift from Dr. Mark Kelley), which have low levels of endogenous APE/ref-1.

Crude extracts containing the overexpressed fusion protein will be purified by, for example, passage over an affinity matrix column (glutathione agarose) that binds the glutathione S-transferase, allowing other proteins to pass through the column. Reduced glutathione is then used to dislodge the fusion protein from the column, to yield a pure fusion protein. This GST-APE/ref-1 fusion protein will then be cleaved with factor Xa as otherwise generally described in A. Yacoub et al., “The DNA repair activity of human redox/repair protein APE/ref-1 is inactivated by phosphorylation,” Cancer Res., vol.: 57, pp. 5457-5459 (1997). The result of this cleavage is liberated APE/ref-1 protein, which is then used in subsequent studies.

### Example 6

Generation of anti-APE/ref-1 antibodies

After the phosphorylation sites are confirmed as discussed in Example 5, standard techniques will be used to generate peptides roughly 10-15 amino acids in length, peptides that are identical to the region surrounding one of the phosphorylation sites of the native APE/ref-1 protein. These peptides will be generated in both phosphorylated and unphosphorylated forms.

The peptides will be conjugated at the carboxy terminus to a cysteine residue to facilitate coupling of the peptide hapten to Sepharose by standard means. This coupled antigen will then be used to generate phospho-specific and non-specific anti-APE/ref-1 antibodies through standard means known in the art. Initially, polyclonal antibodies will be made, but monoclonal antibodies may also be generated through standard means known in the art. Briefly, crude serum is absorbed onto Protein G Sepharose, and eluted under low pH conditions. The resulting fraction is dialyzed, and absorbed onto a non-phosphorylated peptide column to bind and remove non-phospho-specific antibodies. This fraction is then affinity-purified by absorption onto a phospho-peptide column, and eluted under low pH conditions. The specificity of the resulting polyclonal antibodies will be confirmed by standard immunoassays, such as ELISA, with phosphorylated and unphosphorylated APE/ref-1 peptides.

Phospho-specific antibodies will be produced. Although the antibodies had not yet been produced as of the filing date of this application, no significant difficulties are expected. The rationale for this expectation is that phosphorylation induces a marked change in redox activity, consistent with a conformational change in the APE/ref-1 protein that should be manifested as distinguishable antigens. If the initial approach to preparing antibodies using altered peptides does encounter difficulties, then antibodies in rabbits will instead be prepared against fusion proteins that contain the region or regions of phosphorylation.

In other contexts and for other purposes, there are a number of prior reports demonstrating that immunoassays may be used to distinguish between phosphorylated and unphosphorylated versions of the same protein. See, e.g., G. Lonart et al., “Characterization of rabphilin phosphorylation using phospho-specific antibodies,” Neuropharmacology, vol. 41, pp. 643-649 (2001); M. G. Callow et al., “Requirement for PAK4 in the anchorage—independent growth of human cancer cell lines,” J. Biol. Chem., vol. 277, pp. 550-558 (2001); T. Buschmann et al., “Jun NH2-terminal kinase phosphorylation of p53 on Thr-81 is important for p53 stabilization and transcriptional activities in response to stress,” Mol. Cell Biol., vol. 21, pp. 2743-2754 (2001); M. Gater, et al., “Ataxia telangiectasia mutated (ATM) kinase and ATM and Rad3 related kinase mediate phosphorylation of Brca1 at distinct and overlapping sites—In vivo assessment using phospho-specific antibodies,” J. Biol. Chem., vol 276, pp.17276-17280 (2001); and J. P. Blaydes et al., “The development and use of phospho-specific-antibodies to study protein phosphorylation,” Methods Mol. Biol., vol 99, pp. 177-189 (2000).

### EXAMPLE 7

Evaluation of Tissue Samples

The incidence of lack of phosphorylation of APE/ref-1 will be examined both in archived breast cancer tissue samples, and in biopsies from breast cancer patients and control patients. The absence of the phosphorylated form of APE/ref-1 in samples from breast cancer patients will be correlated with clinical observations such as size, grade, and nodule status to verify the accuracy of phosphorylated APE/ref-1 as an indicator for breast cancer.

To conduct immunohistochemistry studies, tissue sections are coated with anti-APE/ref-1 antibody; e.g., mouse anti-APE/ref-1 monoclonal, or rabbit anti-human APE/ref-1 polyclonal, or the phospho-specific antibodies described above. The tissue samples will be incubated overnight at 4° C. at a pre-determined dilution in 10% goat serum in PBS. The following day, sections are washed three times for five minutes in PBS, and are then incubated with biotinylated goat anti-mouse or goat anti-rabbit IgG at 15 μg/mL in 10%; goat serum for one hour. After two PBS washes for 5 minutes each, the sections are incubated in avidin and biotinylated horseradish peroxidase complex for 45 minutes. The sections are then incubated with diaminobenzidine. After sufficient time for the development of a color signal, slides with the sections are washed in distilled water, counter-stained with eosin, dehydrated through a graded alcohol-to-xylene sequence, cover-slipped, analyzed, and photographed. As a negative control, pre-immune IgG (50 μg/mL) is used as the primary antibody in lieu of the anti-APE/ref-1 antibody.

Fixed paraffin-embedded tissue sections are examined for APE/ref-1 using immunohistochemical techniques, employing either commercially available anti-APE/ref-1 polyclonal antibodies (Novus, Littleton, Colo.), or monoclonal antibodies generated for this purpose using standard means known in the art, or the phospho-specific antibodies described above.

### EXAMPLE 9

Tracking the Progression of Phosphorylation Status in Animal Models

In animal models, such as a murine or canine line that is genetically susceptible to breast cancer, the phosphorylation status of APE/ref-1 in breast cancers will be traced, beginning at original diagnosis, and continuing during various types of treatment, to validate measurements of the phosphorylation status of APE/ref-1 as a valuable diagnostic test in breast cancers.

### EXAMPLE 10

Transformed Mice Lacking APE/ref-1 Phosphorylation Site(s)

Using standard means of genetic transformation known in the art, a dominant/negative line of mice will be created with a mutation in one or more APE/ref-1 sites where phosphorylation normally occurs. The transgenic mice will be challenged with DNA-damaging agents (e.g., ionizing radiation or chemical mutagens known in the art) to demonstrate that lack of phosphorylation induces a higher pre-disposition to breast cancer, as one would expect consistent with the findings reported here.

Miscellaneous

Although the initial studies reported here were conducted with breast cancer cells, this invention is also expected to work in diagnosing ovarian cancers, as both ovarian and breast cancers appear to be are characterized by unusual levels of APE/ref-1 in the cytoplasm.

The complete disclosures of all references cited in this specification are hereby incorporated by reference. Also incorporated by reference are the entire texts of the following grant application and the following abstract, both of which are believed to have been unpublished as of the provisional priority date of application Ser. No. 60/360,488, and neither of which is believed to be prior art to this application: W. A. Deutsch, “Phosphorylation Status of APE/Ref-1 Redox/Repair Protein as a Biomarker for Breast Cancer,” Clinical Bridge Award Proposal submitted to the Department of Defense (June 2001); and W. A. Deutsch et al., “The phosphorylation of the multifunctional DNA repair/redox protein Ape1/ref-1 is absent in breast cancer cell lines,” abstract submitted on Nov. 9, 2001 for the April 2002 93rd Annual Meeting of the American Association for Cancer Research. In the event of an otherwise irreconcilable conflict, however, the present specification shall control.

I claim:

1. A method for diagnosing breast cancer or ovarian cancer in a patient, said method comprising assaying the patient for breast cells or ovarian cells showing an absence of phosphorylated APE/ref-1, wherein cells showing an absence of phosphorylated APE/ref-1 indicate a likelihood of cancer.

2. A method as recited in claim 1, wherein said method comprises assaying breast cells.

3. A method as recited in claim 1, wherein said method comprises assaying ovarian cells.

4. A method as recited in claim 1, wherein said assaying comprises an immunoassay with one or more antibodies specific to phosphorylated APE/ref-1.

5. A method as recited in claim 1, wherein said assaying comprises an immunoassay with one or more antibodies specific to APE/ref-1 phosphorylated at amino acid 26.

6. A method as recited in claim 1, wherein said assaying comprises an immunoassay with one or more antibodies specific to APE/ref-1 phosphorylated at amino acid 45.

7. A method as recited in claim 1, wherein said assaying comprises an immunoassay with one or more antibodies specific to APE/ref-1 phosphorylated at amino acid 54.

8. A method as recited in claim 1, wherein said assaying comprises an immunoassay with one or more antibodies specific to APE/ref-1 phosphorylated at amino acid 56.

9. A method as recited in claim 1, wherein said assaying comprises an immunoassay with one or more antibodies specific to APE/ref-1 phosphorylated at amino acid 61.

10. A method as recited in claim 1, wherein said assaying comprises an immunoassay with one or more antibodies specific to APE/ref-1 phosphorylated at amino acid 123.

11. A method as recited in claim 1, wherein said assaying comprises an immunoassay with one or more antibodies specific to APE/ref-1 phosphorylated at amino acid 128.

12. A method as recited in claim 1, wherein said assaying comprises an immunoassay with one or more antibodies specific to APE/ref-1 phosphorylated at amino acid 201.

13. A method as recited in claim 1, wherein said assaying comprises an immunoassay with one or more antibodies specific to APE/ref-1 phosphorylated at amino acid 233.

14. A method as recited in claim 1, wherein said assaying comprises an immunoassay with one or more antibodies specific to APE/ref-1 phosphorylated at amino acid 252.

15. A method as recited in claim 1, wherein said assaying comprises an immunoassay with one or more antibodies specific to APE/ref-1 phosphorylated at amino acid 257.

16. A method as recited in claim 1, wherein said assaying comprises an immunoassay with one or more antibodies specific to APE/ref-1 phosphorylated at amino acid 260.

17. A method as recited in claim 1, wherein said assaying comprises an immunoassay with one or more antibodies specific to APE/ref-1 phosphorylated at amino acid 262.

18. A method as recited in claim 1, wherein said assaying comprises an immunoassay with one or more antibodies specific to APE/ref-1 phosphorylated at amino acid 298.

19. Antibodies against APE/ref-1, wherein said antibodies are specific to phosphorylated APE/ref-1 or are specific to unphosphorylated APE/ref-1.

20. Antibodies as recited in claim 19, wherein said antibodies are specific to phosphorylated APE/ref-1.

21. Antibodies as recited in claim 19, wherein said antibodies are polyclonal.

22. Antibodies as recited in claim 19, wherein said antibodies are monoclonal.

23. Antibodies as recited in claim 19, wherein said antibodies are specific to APE/ref-1 phosphorylated at amino acid 26.

24. Antibodies as recited in claim 19, wherein said antibodies are specific to APE/ref-1 phosphorylated at amino acid 45.

25. Antibodies as recited in claim 19, wherein said antibodies are specific to APE/ref-1 phosphorylated at amino acid 54.

26. Antibodies as recited in claim 19, wherein said antibodies are specific to APE/ref-1 phosphorylated at amino acid 56.

27. Antibodies as recited in claim 19, wherein said antibodies are specific to APE/ref-1 phosphorylated at amino acid 61.

28. Antibodies as recited in claim 19, wherein said antibodies are specific to APE/ref-1 phosphorylated at amino acid 123.

29. Antibodies as recited in claim 19,.,wherein said antibodies are specific to APE/ref-1 phosphorylated at amino acid 128.

30. Antibodies as recited in claim 19, wherein said antibodies are specific to APE/ref-1 phosphorylated at amino acid 201.

31. Antibodies as recited in claim 19, wherein said antibodies are specific to APE/ref-1 phosphorylated at amino acid 233.

32. Antibodies as recited in claim 19, wherein said antibodies are specific to APE/ref-1 phosphorylated at amino acid 252.

33. Antibodies as recited in claim 19, wherein said antibodies are specific to APE/ref-1 phosphorylated at amino acid 257.

34. Antibodies as recited in claim 19, wherein said antibodies are specific to APE/ref-1 phosphorylated at amino acid 260.

35. Antibodies as recited in claim 19, wherein said antibodies are specific to APE/ref-1 phosphorylated at amino acid 262.

36. Antibodies as recited in claim 19, wherein said antibodies are specific to APE/ref-1 phosphorylated at amino acid 298.

37. A method as recited in claim 1, wherein said assaying comprises an immunoassay with one or more antibodies specific to unphosphorylated APE/ref-1.

38. A method as recited in claim 1, wherein said assaying comprises an immunoassay with one or more antibodies specific to APE/ref-1 unphosphorylated at amino acid 26.

39. A method as recited in claim 1, wherein said assaying comprises an immunoassay with one or more antibodies specific to APE/ref-1 unphosphorylated at amino acid 45.

40. A method as recited in claim 1, wherein said assaying comprises an immunoassay with one or more antibodies specific to APE/ref-1 unphosphorylated at amino acid 54.

41. A method as recited in claim 1, wherein said assaying comprises an immunoassay with one or more antibodies specific to APE/ref-1 unphosphorylated at amino acid 56.

42. A method as recited in claim 1, wherein said assaying comprises an immunoassay with one or more antibodies specific to APE/ref-1 unphosphorylated at amino acid 61.

43. A method as recited in claim 1, wherein said assaying comprises an immunoassay with one or more antibodies specific to APE/ref-1 unphosphorylated at amino acid 123.

44. A method as recited in claim 1, wherein said assaying comprises an immunoassay with one or more antibodies specific to APE/ref-1 unphosphorylated at amino acid 128.

45. A method as recited in claim 1, wherein said assaying comprises an immunoassay with one or more antibodies specific to APE/ref-1 unphosphorylated at amino acid 201.

46. A method as recited in claim 1, wherein said assaying comprises an immunoassay with one or more antibodies specific to APE/ref-1 unphosphorylated at amino acid 233.

47. A method as recited in claim 1, wherein said assaying comprises an immunoassay with one or more antibodies specific to APE/ref-1 unphosphorylated at amino acid 252.

48. A method as recited in claim 1, wherein said assaying comprises an immunoassay with one or more antibodies specific to APE/ref-1 unphosphorylated at amino acid 257.

49. A method as recited in claim 1, wherein said assaying comprises an immunoassay with one or more antibodies specific to APE/ref-1 unphosphorylated at amino acid 260.

50. A method as recited in claim 1, wherein said assaying comprises an immunoassay with one or more antibodies specific to APE/ref-1 unphosphorylated at amino acid 262.

51. A method as recited in claim 1, wherein said assaying comprises an immunoassay with one or more antibodies specific to APE/ref-1 unphosphorylated at amino acid 298.

52. Antibodies as recited in claim 19, wherein said antibodies are specific to unphosphorylated APE/ref-1.

53. Antibodies as recited in claim 19, wherein said antibodies are specific to APE/ref-1 unphosphorylated at amino acid 26.

54. Antibodies as recited in claim 19, wherein said antibodies are specific to APE/ref-1 unphosphorylated at amino acid 45.

55. Antibodies as recited in claim 19, wherein said antibodies are specific to APE/ref-1 unphosphorylated at amino acid 54.

56. Antibodies as recited in claim 19, wherein said antibodies are specific to APE/ref-1 unphosphorylated at amino acid 56.

57. Antibodies as recited in claim 19, wherein said antibodies are specific to APE/ref-1 unphosphorylated at amino acid 61.

58. Antibodies as recited in claim 19, wherein said antibodies are specific to APE/ref-1 unphosphorylated at amino acid 123.

59. Antibodies as recited in claim 19, wherein said antibodies are specific to APE/ref-1 unphosphorylated at amino acid 128.

60. Antibodies as recited in claim 19, wherein said antibodies are specific to APE/ref-1 unphosphorylated at amino acid 201.

61. Antibodies as recited in claim 19, wherein said antibodies are specific to APE/ref-1 unphosphorylated at amino acid 233.

62. Antibodies as recited in claim 19, wherein said antibodies are specific to APE/ref-1 unphosphorylated at amino acid 252.

63. Antibodies as recited in claim 19, wherein said antibodies are specific to APE/ref-1 unphosphorylated at amino acid 257.

64. Antibodies as recited in claim 19, wherein said antibodies are specific to APE/ref-1 unphosphorylated at amino acid 260.

65. Antibodies as recited in claim 19, wherein said antibodies are specific to APE/ref-1 unphosphorylated at amino acid 262.

66. Antibodies as recited in claim 19, wherein said antibodies are specific to APE/ref-1 unphosphorylated at amino acid 298.


SEQUENCE LISTING (1 sequence)

SEQ ID NO: 1 - protein, 318 aa, Homo sapiens
MPKRGKKGAVAEDGDELRTEPEAKKSKTAAKKNDKEAAGEGPALYEDPPDQKTSPSGKPATLKICSWNVDGLRAWIKKKGLDWVKEEAPDILCLQETKCSENKLPAELQELPGLSHQYWSAPSDKEGYSGVGLLSRQCPLKVSYGIGDEEHDQEGRVIVAEFDSFVLVTAYVPNAGRGLVRLEYRQRWDEAFRKFLKGLASRKPLVLCGDLNVAHEEIDLRNPKGNKKNAGFTPQERQGFGELLQAVPLADSFRHLYPNTPYAYTFWTYMMNARSKNVGWRLDYFLLSHSLLPALCDSKIRSKALGSDHCPITLYLAL